SEQUENCE LISTING (6 sequences)

SEQ ID NO: 1 - DNA, 1242 bp, Saccharomyces cerevisiae
  source 1..1242: mol_type = genomic DNA; note = MRF1
atgtggctttcaaagttccagttcccctcgaggtccatctttaagggtgtttttttgggtcacaagttgccgttactagtgcgactgacatcaacaacgactaactctaagagcaacggcagcatcccaacacaatacacagaactctctccgttacttgttaagcaagcggaaaagtatgaagctgaactaaaagatcttgacaaagacctttcttgcggcattcattttgacgtgaataagcagaaacattatgctaaattatcagcgctcactgatacatttattgagtataaggaaaaactaaatgaattgaaaagtttacaagaaatgattgtatctgatccatcattaagggcggaggctgaacaagaatatgcagaactggtcccccaatatgaaacgacctcctcaagattggtaaataaacttcttccaccgcatccgttcgcagataaacctagcttactagagcttcgaccgggtgtaggtggcattgaagccatgatttttacccagaatctattggatatgtatattggctatgccaactatagaaaatggaagtatcgaattatatcgaaaaatgaaaacgaaagtgggtcaggcattatcgatgccattctgagcattgaagaagccggatcttacgatcgtttgaggtttgaagcaggcgttcacagggtgcagagaattcctagtacggagactaaaggaaggacgcacacatccactgctgccgtggttgtgttacctcaaattggtgacgaatctgctaaatctattgacgcttatgaaagaacatttaaacctggtgaaatcagagttgacatcatgcgtgcaagcgggaaaggtggtcaacatgtgaatacgacagactctgctgtcagattaacccatatcccctctggaattgttgtttccatgcaagatgaaagatctcaacacaagaataaggctaaagcatttacgatcttaagagcgagacttgcagagaaggaaaggctggaaaaggaggaaaaagaaagaaaagcaagaaagagtcaggtttctagcaccaataggtctgataaaattagaacatacaattttccacagaacagaatcactgatcataggtgcgggttcacattgttggacctgccaggtgtgttatctggagaacgattggacgaagttattgaggcaatgtctaaatatgatagcaccgaacgggcaaaagaattattggagagtaactga

SEQ ID NO: 2 - protein, 413 aa, Saccharomyces cerevisiae
MWLSKFQFPSRSIFKGVFLGHKLPLLVRLTSTTTNSKSNGSIPTQYTELSPLLVKQAEKYEAELKDLDKDLSCGIHFDVNKQKHYAKLSALTDTFIEYKEKLNELKSLQEMIVSDPSLRAEAEQEYAELVPQYETTSSRLVNKLLPPHPFADKPSLLELRPGVGGIEAMIFTQNLLDMYIGYANYRKWKYRIISKNENESGSGIIDAILSIEEAGSYDRLRFEAGVHRVQRIPSTETKGRTHTSTAAVVVLPQIGDESAKSIDAYERTFKPGEIRVDIMRASGKGGQHVNTTDSAVRLTHIPSGIVVSMQDERSQHKNKAKAFTILRARLAEKERLEKEEKERKARKSQVSSTNRSDKIRTYNFPQNRITDHRCGFTLLDLPGVLSGERLDEVIEAMSKYDSTERAKELLESN

SEQ ID NO: 3 - DNA, 930 bp, Saccharomyces cerevisiae
  source 1..930: mol_type = genomic DNA; note = YOR292C
atgcctcttcaactttttggcagggatcagatcgttgtacattatgataacggcaacatgagtaacgatgaccagaatcaccaaagtgttctcggatcttggacaagaagggcagcagcagcattaagaacgttaatgaacaagcgaatacaacgtataacacttacgcactggttgctgttagtcatatgggttactagcctatggaaatttaccagtcattatagacagctgtatgctaattctgctgtttttgcaactttatgcacgaacattcttttatttgggatatctgacatactagcacaaagtatagcctgcttttattcatatcacgtggaccctataccgcaaatacttaacgatacctttcatcatgttcagaacaaccgtgatgtagaaaatggaggaggttatgaaagtgatgagctatctatcttcaatgattttacctcggaacatagctcctacacggataacgacgactatcctgaacttgataggccattagctactttcaaaacggacacatttgacttcttcaggtgggggtgttttatgttctggggattttttatatcatttttccaggctccttggtacaagttcttgaatttcttttacactgaagatccgacagtggtacaggtttttgaaagggttctttcggatcagctcctttattcgccaatctctctctattgtttctttatgttctcaaactatgtgatggaaggtggagataaggacacccttggcaagaaaatccaaaggctatatatatctacgcttggttgtaactatctggtgtggccaatggtacaattcattaatttccttataatgcctagagatttccaagcaccttttagctcttcagttggagtggtatggaattgcttcctttcgatgagaaatgcgtctaaatag

SEQ ID NO: 4 - protein, 309 aa, Saccharomyces cerevisiae
MPLQLFGRDQIVVHYDNGNMSNDDQNHQSVLGSWTRRAAAALRTLMNKRIQRITLTHWLLLVIWVTSLWKFTSHYRQLYANSAVFATLCTNILLFGISDILAQSIACFYSYHVDPIPQILNDTFHHVQNNRDVENGGGYESDELSIFNDFTSEHSSYTDNDDYPELDRPLATFKTDTFDFFRWGCFMFWGFFISFFQAPWYKFLNFFYTEDPTVVQVFERVLSDQLLYSPISLYCFFMFSNYVMEGGDKDTLGKKIQRLYISTLGCNYLVWPMVQFINFLIMPRDFQAPFSSSVGVVWNCFLSMRNASK

SEQ ID NO: 5 - DNA, 366 bp, Saccharomyces cerevisiae
  source 1..366: mol_type = genomic DNA; note = YGR190C
atgtcttcctgctctgtgtcttcattacgacggaagcgatctgaagaggtaatatcgtttgtggtttggtttatgccaggtgaatccttgctacttaatggaaatgcagaatcggttttggaagttagttcggtaacaaatgtagtagattctttttcattctcgaaggctggagaagtagccggcttgaatccgtcaactacatctgcccaatattcctttttcaatgggtttctaggcatttttgattttgaggttgtatatctgtatttgtgtactaatgagtcagatagcgataatattatacgtcaaaaaaacttgggaaggacaatgaattgtaataacgctgatgatatgttgttttga

SEQ ID NO: 6 - protein, 121 aa, Saccharomyces cerevisiae
MSSCSVSSLRRKRSEEVISFVVWFMPGESLLLNGNAESVLEVSSVTNVVDSFSFSKAGEVAGLNPSTTSAQYSFFNGFLGIFDFEVVYLYLCTNESDSDNIIRQKNLGRTMNCNNADDMLF

(19) United States
(12) Patent Application Publication
(10) Pub. No.: US 2024/0279287 A1
(43) Pub. Date: Aug. 22, 2024

(54) GENETICALLY MODIFIED YEAST AND FERMENTATION MIXTURES AND METHODS USING GENETICALLY MODIFIED YEAST

(71) Applicant: John I. Haas, Inc., Washington, DC (US)

(72) Inventors: Sami Faour, Washington, DC (US); Michail Karavolos, Great Malvern (GB)

(21) Appl. No.: 18/440,744

(22) Filed: Feb. 13, 2024

Related U.S. Application Data
(60) Provisional application No. 63/485,498, filed on Feb. 16, 2023.

(51) Int. Cl. C07K 14/395; C12P 7/06

(52) U.S. Cl. CPC C07K 14/395; C12P 7/06

(57) ABSTRACT

The present disclosure provides a genetically modified yeast strain comprising a S. cerevisiae comprising a modification of at least one gene encoding mitochondrial release factor 1 (MRF1), YOR292C, or YGR190C, wherein the modification disrupts expression of a protein from the gene. The present disclosure further provides a fermentation mixture comprising a genetically modified yeast strain and an organic material for fermentation and, in some embodiments, a hop acid. The present disclosure further provides a method of fermenting such as fermentation mixture.

### PRIORITY CLAIM

The present application claims priority under 35 U.S.C. § 119 to U.S. Provisional Patent Application Ser. No. 63/485,498, filed Feb. 16, 2023 and titled “GENETICALLY MODIFIED YEAST AND FERMENTATION MIXTURES AND METHODS USING GENETICALLY MODIFIED YEAST,” which is incorporated by reference herein in its entirety.

### TECHNICAL FIELD

The present disclosure provides genetically modified yeast cells, fermentation mixtures containing genetically modified yeast and organic materials, hop acids, or both, and methods of fermenting organic materials using the genetically modified yeast in the presence of hop acids.

### BACKGROUND

Fermentation of organic materials by yeast to product ethanol has may applications, including the industrial production of ethanol. Hop acids are frequently added to fermentation mixtures to control bacterial growth. Bacterial grown in fermentation mixtures decreases ethanol production because bacteria compete with yeast for organic materials, which are used as food by both types of organisms, but bacteria, unlike yeast, bacteria do not produce ethanol. Bacteria may also produce lactic acid, which is undesirable in most fermentations.

### BRIEF SUMMARY

The present disclosure provides a genetically modified yeast strain comprising a S. cerevisiae comprising a modification of at least one gene encoding mitochondrial release factor 1 (MRF1), YOR292C, or YGR190C, wherein the modification disrupts expression of a protein from the gene.

In specific embodiments, which may be combined with one another or any other aspects of this specification unless clearly mutually exclusive:
the modification is of a nucleic acid sequence of the gene;
the modification comprises an insertion into a coding portion of the gene;
the modification comprises a deletion of all or a portion of the gene;
the modification is not of a nucleic acid sequence of the gene;
the S. cerevisiae comprises a modification of at least two genes encoding MRF1, YOR292C, or YGR190C;
the S. cerevisiae comprises a modification of all three genes encoding MRF1, YOR292C, and YGR190C;
a gene encoding MRF1, absent modification, has a nucleic acid sequence at least 90% identical to that of SEQ ID NO: 1, or encodes a protein having an amino acid sequence at least 90% identical to that of SEQ ID NO: 2;
the gene encoding MRF1, absent modification, has a nucleic acid sequence of SEQ ID NO: 1, or encodes a protein having an amino acid sequence of SEQ ID NO: 2;
the gene encoding YOR292C, absent modification, has a nucleic acid sequence at least 90% identical to that of SEQ ID NO: 3, or encodes a protein having an amino acid sequence at least 90% identical to that of SEQ ID NO: 4;
the gene encoding YOR292C, absent modification, has a nucleic acid sequence of SEQ ID NO: 3, or encodes a protein having an amino acid sequence of SEQ ID NO: 4;
the gene encoding YGR190C, absent modification, has a nucleic acid sequence at least 90% identical to that of SEQ ID NO: 5, or encodes a protein having an amino acid sequence at least 90% identical to that of SEQ ID NO: 6;
the gene encoding YGR190C, absent modification, has a nucleic acid sequence of SEQ ID NO: 5, or encodes a protein having an amino acid sequence of SEQ ID NO: 6;
the genetically modified yeast strain comprises an ER yeast strain comprising a modification in the gene encoding YGR190C;
the genetically modified yeast strain comprises a PE-2 yeast strain comprising a modification in the gene encoding YOR292C;
the genetically modified yeast strain comprises a PE-2 yeast strain comprising a modification in the gene encoding MRF1.

The present disclosure further provides a fermentation mixture comprising a genetically modified yeast strain as described above or elsewhere in the specification; an organic material for fermentation; and a hop acid.

In specific embodiments, which may be combined with one another or any other aspects of this specification unless clearly mutually exclusive:
the hop acid comprises an alpha acid or an iso-alpha acid.
the hop acid comprises a beta acid.
the hop acid comprises a tetra-hydro iso-alpha acid (THIAA), a hexa-hydro iso-alpha acid (HHIAA), or a combination thereof.

The present disclosure further provides a fermentation mixture comprising: a genetically modified yeast strain as described above or elsewhere in the specification; and an organic material for fermentation.

The present disclosure provides a fermentation method comprising fermenting a fermentation mixture comprising hop acids as described above or elsewhere in the specification, for a time sufficient to produce between 0.5% and 4% more ethanol than fermenting an otherwise identical fermentation mixture lacking the hop acid.

The present disclosure provides a fermentation method comprising fermenting a fermentation mixture comprising hop acids as described above or elsewhere in the specification, for a time sufficient to produce more ethanol than fermenting an otherwise identical fermentation mixture using an unmodified native yeast strain that was modified to produce the genetically modified yeast strain.

The present disclosure provides a fermentation method comprising fermenting a fermentation mixture lacking a hop acid as described above or elsewhere in the specification, for a time sufficient to produce more ethanol than fermenting an otherwise identical fermentation mixture using an unmodified native yeast strain that was modified to produce the genetically modified yeast strain.

### DETAILED DESCRIPTION

The present disclosure provides genetically modified yeast cells, fermentation mixtures containing genetically modified yeast and organic materials, hop acids, or both, and methods of fermenting organic materials using the genetically modified yeast in the presence of hop acids. “Yeast,” unless defined as a given species or strain, refers to any member of the phylum Ascomycota or class Saccharomyces.

“Genetically modified yeast” refers to yeast that have had their genetic material altered by an exogenous factor, which refers to any substance not natively found within the yeast cell or to processes in the yeast cell resulting from exposure to such as substance. Although the present disclosure discusses particular genetic modifications in detail, genetically modified yeast as disclosed herein may contain additional genetic modifications.

As used herein “variant yeast” or a “variant strain” refer to genetically modified yeast that are genetically identical (with the exception of random mutations that occasionally occur during yeast culture or fermentation, and that are not present in a substantial number of the yeast in such culture or fermentation mixture).

“Native yeast” refers to yeast strains that are not genetically modified yeast

A “hop acid” is an organic acid produced in the cones of hop plants, and isomers or derivatives thereof. Hop acids include “alpha acids,” also referred to as “humulones” and “beta acids” also referred to as “lupulones.” “Hop acids” also include cis and trans-iso-α-acids, cis and trans-p-iso-α-acids, tetrahydrodesoxy-α-acids, tetrahydro-α-acids, and cis and trans-tetrahydroiso α-acids. Hop acids that are derivatives of those found in hops may be prepared using various techniques. For example, alpha acids may be heat-treated to form “iso-alpha acids,” which are isomerized forms.

The term “about,” as used herein, refers to a variation of 5% or less. The term “or,” as used herein, unless clearly indicated by context to refer to only one alternative, is used in the inclusive sense, equivalent to “and/or.”

### Genetically Modified Yeast

The present disclosure provides genetically modified yeast that exhibit an increase in ethanol production during fermentation in the presence of hop acids. In some embodiments, the genetically modified yeast exhibit increased ethanol production during fermentation in the present of alpha acids or iso-alpha acids. In some embodiments, the genetically modified yeast exhibit increased ethanol production during fermentation in the presence of beta acids. In some embodiments, the genetically modified yeast exhibit increase ethanol production during fermentation in the presence of both alpha acids and beta acids.

Genetically modified yeast of the present disclosure are modified to cease production of at least one specific protein. Typically, the genetically modified yeast contain a modification of the sequence of the gene encoding such a protein.

In some embodiments, the sequence of the coding portion of the gene itself is modified. In some specific embodiments, the coding portion is deleted. In other embodiments, the coding portion is partially deleted, such that a functional protein may be not be produced. In other embodiments, the coding portion is modified to prevent production of a functional protein, for example by introduction of a stop codon or a mutation that prevents proper protein folding or activity. In still other embodiments, the coding portion of the gene is disrupted by an insertion, such as insertion of another gene. For example, a kanamycin cassette, such as that described in the Examples, may be inserted.

In some embodiments, the sequence of a regulatory portion of the gene is modified. For example, the promoter may be deleted or disrupted.

In some embodiments, the sequences of the coding portion and of a regulatory portion of the gene may both be modified.

In some embodiments, the entire gene is deleted.

In some embodiments, the sequence of the gene is not modified, but other modifications, such as methylation changes or nucleotide inactivation, are used to prevent expression of functional protein.

Genetically modified yeast of the present disclosure may be derived from native yeast of any species or strain. In specific embodiments, the species or strain is one that produces ethanol during fermentation, such as S. cerevisiae. Yeast of the present invention may be directly derived from native yeast, or they may be derived from genetically modified yeast containing modifications other than those described herein. Yeast of the present invention may also be further genetically modified.

Genetically modified yeast of the present disclosure contain a modification of the gene encoding at least one of mitochondrial release factor 1 (MRF1), YOR292C (a vacuole-localizing protein with undefined function), or YGR190C (a protein with undefined function). In some embodiments, the genetically modified yeast contains a modification of only one of these genes. In some embodiments, the genetically modified yeast contains a modification of two of these genes. In some embodiments, the genetically modified yeast contains a modification of all three of these genes.

MRF1 is the gene identified in the Saccharomyces Genome Database (SGD), as SGD:S000003111, located at Chromosome VII 234717 . . . 235958. MRF1 has the nucleic acid sequence:

| (SEQ ID NO: 1) | |
|---|---|
| 1 | ATGTGGCTTT CAAAGTTCCA GTTCCCCTCG AGGTCCATCT TTAAGGGTGT TTTTTTGGGT |
| 61 | CACAAGTTGC CGTTACTAGT GCGACTGACA TCAACAACGA CTAACTCTAA GAGCAACGGC |
| 121 | AGCATCCCAA CACAATACAC AGAACTCTCT CCGTTACTTG TTAAGCAAGC GGAAAAGTAT |
| 181 | GAAGCTGAAC TAAAAGATCT TGACAAAGAC CTTTCTTGCG GCATTCATTT TGACGTGAAT |
| 241 | AAGCAGAAAC ATTATGCTAA ATTATCAGCG CTCACTGATA CATTTATTGA GTATAAGGAA |
| 301 | AAACTAAATG AATTGAAAAG TTTACAAGAA ATGATTGTAT CTGATCCATC ATTAAGGGCG |
| 361 | GAGGCTGAAC AAGAATATGC AGAACTGGTC CCCCAATATG AAACGACCTC CTCAAGATTG |
| 421 | GTAAATAAAC TTCTTCCACC GCATCCGTTC GCAGATAAAC CTAGCTTACT AGAGCTTCGA |
| 481 | CCGGGTGTAG GTGGCATTGA AGCCATGATT TTTACCCAGA ATCTATTGGA TATGTATATT |
| 541 | GGCTATGCCA ACTATAGAAA ATGGAAGTAT CGAATTATAT CGAAAAATGA AAACGAAAGT |
| 601 | GGGTCAGGCA TTATCGATGC CATTCTGAGC ATTGAAGAAG CCGGATCTTA CGATCGTTTG |
| 661 | AGGTTTGAAG CAGGCGTTCA CAGGGTGCAG AGAATTCCTA GTACGGAGAC TAAAGGAAGG |
| 721 | ACGCACACAT CCACTGCTGC CGTGGTTGTG TTACCTCAAA TTGGTGACGA ATCTGCTAAA |
| 781 | TCTATTGACG CTTATGAAAG AACATTTAAA CCTGGTGAAA TCAGAGTTGA CATCATGCGT |
| 841 | GCAAGCGGGA AAGGTGGTCA ACATGTGAAT ACGACAGACT CTGCTGTCAG ATTAACCCAT |
| 901 | ATCCCCTCTG GAATTGTTGT TTCCATGCAA GATGAAAGAT CTCAACACAA GAATAAGGCT |
| 961 | AAAGCATTTA CGATCTTAAG AGCGAGACTT GCAGAGAAGG AAAGGCTGGA AAAGGAGGAA |
| 1021 | AAAGAAAGAA AAGCAAGAAA GAGTCAGGTT TCTAGCACCA ATAGGTCTGA TAAAATTAGA |
| 1081 | ACATACAATT TTCCACAGAA CAGAATCACT GATCATAGGT GCGGGTTCAC ATTGTTGGAC |
| 1141 | CTGCCAGGTG TGTTATCTGG AGAACGATTG GACGAAGTTA TTGAGGCAAT GTCTAAATAT |
| 1201 | GATAGCACCG AACGGGCAAA AGAATTATTG GAGAGTAACT GA |

and encodes a protein with the amino acid sequence:

| (SEQ ID NO: 2) | |
|---|---|
| 1 | MWLSKFQFPS RSIFKGVFLG HKLPLLVRLT STTTNSKSNG SIPTQYTELS PLLVKQAEKY |
| 61 | EAELKDLDKD LSCGIHFDVN KQKHYAKLSA LTDTFIEYKE KLNELKSLQE MIVSDPSLRA |
| 121 | EAEQEYAELV PQYETTSSRL VNKLLPPHPF ADKPSLLELR PGVGGIEAMI FTQNLLDMYI |
| 181 | GYANYRKWKY RIISKNENES GSGIIDAILS IEEAGSYDRL RFEAGVHRVQ RIPSTETKGR |
| 241 | THTSTAAVVV LPQIGDESAK SIDAYERTFK PGEIRVDIMR ASGKGGQHVN TTDSAVRLTH |
| 301 | IPSGIVVSMQ DERSQHKNKA KAFTILRARL AEKERLEKEE KERKARKSQV SSTNRSDKIR |
| 361 | TYNFPQNRIT DHRCGFTLLD LPGVLSGERL DEVIEAMSKY DSTERAKELL ESN* |

YOR292C is the gene identified in the Saccharomyces Genome Database (SGD), as SGD: S000005818, located at Chromosome XV 865653 . . . 866582. YOR292C has the nucleic acid sequence:

| (SEQ ID NO: 3) | |
|---|---|
| 1 | ATGCCTCTTC AACTTTTTGG CAGGGATCAG ATCGTTGTAC ATTATGATAA CGGCAACATG |
| 61 | AGTAACGATG ACCAGAATCA CCAAAGTGTT CTCGGATCTT GGACAAGAAG GGCAGCAGCA |
| 121 | GCATTAAGAA CGTTAATGAA CAAGCGAATA CAACGTATAA CACTTACGCA CTGGTTGCTG |
| 181 | TTAGTCATAT GGGTTACTAG CCTATGGAAA TTTACCAGTC ATTATAGACA GCTGTATGCT |
| 241 | AATTCTGCTG TTTTTGCAAC TTTATGCACG AACATTCTTT TATTTGGGAT ATCTGACATA |
| 301 | CTAGCACAAA GTATAGCCTG CTTTTATTCA TATCACGTGG ACCCTATACC GCAAATACTT |
| 361 | AACGATACCT TTCATCATGT TCAGAACAAC CGTGATGTAG AAAATGGAGG AGGTTATGAA |
| 421 | AGTGATGAGC TATCTATCTT CAATGATTTT ACCTCGGAAC ATAGCTCCTA CACGGATAAC |
| 481 | GACGACTATC CTGAACTTGA TAGGCCATTA GCTACTTTCA AAACGGACAC ATTTGACTTC |
| 541 | TTCAGGTGGG GGTGTTTTAT GTTCTGGGGA TTTTTTATAT CATTTTTCCA GGCTCCTTGG |
| 601 | TACAAGTTCT TGAATTTCTT TTACACTGAA GATCCGACAG TGGTACAGGT TTTTGAAAGG |
| 661 | GTTCTTTCGG ATCAGCTCCT TTATTCGCCA ATCTCTCTCT ATTGTTTCTT TATGTTCTCA |
| 721 | AACTATGTGA TGGAAGGTGG AGATAAGGAC ACCCTTGGCA AGAAAATCCA AAGGCTATAT |
| 781 | ATATCTACGC TTGGTTGTAA CTATCTGGTG TGGCCAATGG TACAATTCAT TAATTTCCTT |
| 841 | ATAATGCCTA GAGATTTCCA AGCACCTTTT AGCTCTTCAG TTGGAGTGGT ATGGAATTGC |
| 901 | TTCCTTTCGA TGAGAAATGC GTCTAAATAG |

and encodes a protein with the amino acid sequence:

| (SEQ ID NO: 4) | |
|---|---|
| 1 | MPLQLFGRDQ IVVHYDNGNM SNDDQNHQSV LGSWTRRAAA ALRTLMNKRI QRITLTHWLL |
| 61 | LVIWVTSLWK FTSHYRQLYA NSAVFATLCT NILLFGISDI LAQSIACFYS YHVDPIPQIL |
| 121 | NDTFHHVQNN RDVENGGGYE SDELSIFNDF TSEHSSYTDN DDYPELDRPL ATFKTDTFDF |
| 181 | FRWGCFMFWG FFISFFQAPW YKFLNFFYTE DPTVVQVFER VLSDQLLYSP ISLYCFFMFS |
| 241 | NYVMEGGDKD TLGKKIQRLY ISTLGCNYLV WPMVQFINFL IMPRDFQAPF SSSVGVVWNC |
| 301 | FLSMRNASK* |

YGR190C is the gene identified in the Saccharomyces Genome Database (SGD), as SGD:S000003422, located at Chromosome VII 880296 . . . 880661. YGR190C has the nucleic acid sequence:

| (SEQ ID NO: 5) | |
|---|---|
| 1 | ATGTCTTCCT GCTCTGTGTC TTCATTACGA CGGAAGCGAT CTGAAGAGGT AATATCGTTT |
| 61 | GTGGTTTGGT TTATGCCAGG TGAATCCTTG CTACTTAATG GAAATGCAGA ATCGGTTTTG |
| 121 | GAAGTTAGTT CGGTAACAAA TGTAGTAGAT TCTTTTTCAT TCTCGAAGGC TGGAGAAGTA |
| 181 | GCCGGCTTGA ATCCGTCAAC TACATCTGCC CAATATTCCT TTTTCAATGG GTTTCTAGGC |
| 241 | ATTTTTGATT TTGAGGTTGT ATATCTGTAT TTGTGTACTA ATGAGTCAGA TAGCGATAAT |
| 301 | ATTATACGTC AAAAAAACTT GGGAAGGACA ATGAATTGTA ATAACGCTGA TGATATGTTG |
| 361 | TTTTGA |

and encodes a protein with the amino acid sequence:

| (SEQ ID NO: 6) | |
|---|---|
| 1 | MSSCSVSSLR RKRSEEVISF VVWFMPGESL LLNGNAESVL EVSSVTNVVD SFSFSKAGEV |
| 61 | AGLNPSTTSA QYSFFNGFLG IFDFEVVYLY LCTNESDSDN IIRQKNLGRT MNCNNADDML |
| 121 | F* |

MRF1, YOR292C, or YGR190C may, in some embodiments, have nucleic acid or protein sequences at least 90%, at least 95%, or at least 99% identical to the nucleic acid or protein sequences disclosed in SEQ ID NOs: 1-6.

In some embodiments of the present disclosure, the genetically modified yeast may produce glucoamylase, either as a native characteristic or as a result of genetic modification.

In a specific embodiments, the genetically modified yeast may be an ER strain modified at YGR190C, such as Variant G. In some embodiments, this genetically modified yeast may exhibit an improvement in ethanol production as compared to the unmodified native ER strain in the presence of hop acids. In some embodiments, this strain may not exhibit an improvement in ethanol production in the absence of hop acids.

In specific embodiments, the genetically modified yeast may be a PE-2 strain modified at YOR292C, such as Variant Q. In some embodiments, this genetically modified yeast may exhibit an improvement in ethanol production as compared to the unmodified native PE-2 strain in the presence of hop acids, in the absence of hop acids, or both in the presence and absence of hop acids.

In specific embodiments, the genetically modified yeast may be a PE-2 strain modified at MRF1, such as Variant W. In some embodiments, this genetically modified yeast may exhibit an improvement in ethanol production as compared to the unmodified native PE-2 strain in the presence of hop acids, in the absence of hop acids, or both in the presence and absence of hop acids.

### Fermentation Mixtures and Methods

The present disclosure also provides a fermentation mixture containing a genetically modified yeast as disclosed herein and an organic material for fermentation, along with at least one hop acid. The disclosure further provides a related method of fermentation using such a fermentation mixture.

In a specific embodiment, the fermentation mixture contains at least 150 ppm of a 30% hop acid solution, such as IsoStab® (BetaTec Hop Products, UK).

In another specific embodiment, the fermentation mixture contains at least 13.5 ppm of a lower concentration hop acid solutions such as LactoStab® (BetaTec Hop Products, UK).

In other specific embodiments, the fermentation mixture contains at least 40 ppm, 45 ppm, 50 ppm, or 60 ppm hop acids at the beginning of fermentation or when hop acids are first added to the fermentation mixture.

In other specific embodiments, the fermentation mixture contains between 40 ppm and 45 ppm, 40 ppm and 50 ppm, 40 ppm and 60 ppm, 45 ppm and 50 ppm, 45 ppm and 60 ppm, or 50 ppm and 60 ppm 60 ppm hop acids at the beginning of fermentation or when hop acids are first added to the fermentation mixture.

In a specific embodiment, the hop acids include alpha acids or iso-alpha acids and beta acids. In another specific embodiment, the hop acids include alpha acids or iso-alpha acids. In another specific embodiment, the hop acids include beta acids.

In some specific embodiments, the hop acids include iso-alpha acids (IAA), such as tetra-hydro iso-alpha acids (THIAA), hexa-hydro iso-alpha acids (HHIAA), or a combination thereof.

In a specific embodiment, the organic material for fermentation is an industrial feedstock material, not intended for eventual human consumption.

In a specific embodiment, the organic material for fermentation includes or consists of a sugar production by-product. In a more specific embodiment, the organic material for fermentation includes or consists of molasses.

In a specific embodiment, the organic material for fermentation includes or consists of corn mash. In a more specific embodiment, the organic material for fermentation includes or consists of whole corn cobs (shucked or unshucked), or corn kernels.

In a specific embodiment, the fermentation mixture is inoculated with approximately 10⁷-10⁸ cfu/ml yeast at the beginning of fermentation. In some embodiments, this amount may vary by as much as 5%, 10%, or 20%.

In some embodiments, the fermentation mixture also contains water.

In some embodiments, the fermentation process, when carried out using a fermentation mixture of the present disclosure, produces more ethanol than an otherwise identical fermentation process using native yeast strains lacking the genetic modifications as disclosed herein. In specific embodiments, ethanol production is increased in a range between 1% and 3%, 1% and 2.9%, 1% and 2.5%, 1% and 2%, 1.4% and 3%, 1.4% and 2.9% 1.4% and 2.5%, 1.4% and 2%, 2% and 3%, 2% and 2.5%, 2% and 2.9%, 2.5% and 3%, or 2.5% and 2.9%.

In some embodiments, the fermentation process is carried out without the presence of hop acids. In such embodiments, the fermentation process, when carried out using a fermentation mixture of the present disclosure, produces more ethanol than an otherwise identical fermentation process using native yeast strains lacking the genetic modifications as disclosed herein. In specific embodiments, ethanol production is increased in a range between 1% and 2.5%, 1% and 2.4%, 1% and 2%, 1% and 1.3%, 1.3% and 2.5%, 1.3% and 2.4%, 1.3% and 2%, 1.3% and 1.5%, 1.5% and 2.5%, 1.5% and 2.4%, 1.5% and 2%, 2% and 2.5%, 2% and 2.4%, or 2.4% and 2.5%.

Fermentation processes of the present disclosure may be carried out in industrial fermenters. Ethanol may be removed from the fermentation mixture at the end of the fermentation process.

### EXAMPLES

### Example 1: Genetically Modified Yeast

S. cerevisiae Ethanol Red strain (ER) or S. cerevisiae PE-2 strain, both of which are commonly used in industrial ethanol production, were genetically modified follows:
Variant G=ER modified at YGR190C
Variant Q=PE-2 modified at YOR292C; and
Variant W=PE-2 modified at MRF1.

Sequence information for the native yeast strains lacking the genetic modifications above may be found at sequence information: Saccharomyces Genome Database|SGD (yeastgenome.org).

Yeast were genetically modified according to the following protocol.
1. Inoculate 5 ml of Yeast Extract-Peptone-Dextrose medium (YPD) with a single yeast colony and grow overnight with shaking at 250 rpm at 30° C.
2. Record OD600 and transfer 4 ml of overnight culture to 100 ml YPD (in 500 ml conical flask). Starting OD600 in 100 ml culture was 0.18.
3. Grow for 3-4 h, check OD600 and start transformation if OD600 is of 0.4-0.6. (Started at 0.48).
4. Centrifuge the cells in 50 ml tubes at 2500 rpm for 5 min at room temperature then pool the pellet.
5. Wash the cells 1× in 20 ml of sterile dH₂O. Centrifuge as above.
6. Wash the cells in 20 ml of freshly prepared 1×Li/TE buffer. Centrifuge as above.
7. Resuspend the cells in 1.2 ml of freshly prepared 1×Li/TE buffer.
8. Incubate for 15 min at 30° C. (During this time denature SS-DNA)
9. Add 200 μl boiled carrier DNA2 to the cells, mix by hand and add 5 ml of PEG¹ solution. Mix again.
10. Add 160 μl of above reaction mixture to 25 μl DNA containing a PCR fragment with the kanamycin cassette and complementary upstream and downstream sites to facilitate recombination with the gene of interest, on 96 well plates. Mix 2 times by pipetting.
11. Incubate for 30 min at 30° ° C. at 250 rpm.
12. Heat shock at 42° C. for 30 min. No DMSO.
13. Chill cells on ice for 30 sec and centrifuge for 5 min at 2000 rpm.
14. Dispose of the supernatant, add 200 μl of YPD and mix.
15. Incubate at room temperature overnight.
16. Plate out on selective media (YPG+300 μg/ml geneticin (Melford)).
17. Incubate upside down at 30° ° C. for 3-4 days.

As control, cells can be plated out on YPG e.g. after steps 4, 8, 10, 11, 13 and 15 to check for growth.

Materials used are as follows: 1 and 2 correspond to superscripts in the above method.
¹PEG: Mixture of 3 ml (30 μl per reaction) cells+12.5 ml PEG and 500 μl of carrier DNA is enough for 100 transformation reactions.
²Carrier DNA: About 15 min before step 9, denature 10 mg/ml of salmon sperm DNA in a PCR machine or in a water bath at 95° C. for 10 min and immediately place on ice.
YPD (1 L): 1% yeast extract (10 g), 2% peptone (20 g), 2% glucose (20 g). Add 1.5% (15 g) agar for plates. Autoclave.
10× LiAc buffer—final 1 M, pH 7.5 (100 ml): Resuspend 10.2 g of lithium acetate dehydrate (MW=102.02 g/mol) in 90 ml of dH₂O. Adjust pH with diluted acetic acid and autoclave.
10× TE buffer—final 100 mM Tris and 10 mM EDTA, pH 7.5 (100 ml): Resuspend 1.21 g of Tris (MW=121.1 g/mol) and 0.29 g EDTA (MW=292.24 g/mol) in 90 ml dH₂O. Adjust pH to 7.5 with HCl and autoclave.
1× Li/TE buffer (50 ml): Mix 5 ml of 10× LiAc buffer with 5 ml 10× TE buffer and fill up to 50 ml with dH₂O.
50% PEG 3350 (100 ml): Dissolve 50 g of PEG 3350 in 70 ml of dH₂O and fill up to 100 ml. Autoclave.
PEG solution—40% PEG 3350 in 1× Li/TE buffer (12.5 ml): Mix: 10 ml of 50% PEG with 1.25 ml of 10× LiAc buffer and 1.25 ml 10× TE buffer.

After the genetic modification procedure, potential genetically modified yeast were plated out for screening using YPD-Kan plates containing Kanamycin antibiotic to select for variants that had their gene replaced by the kanamycin cassette.

Genetic modifications were confirmed by PCR reactions carried out with DNA primers designed according to the DNA gene sequences provided in the Saccharomyces Genome Database (https://www.yeastgenome.org/).

### Example 2: Corn Fermentation

Genetically modified yeast were used in a fermentation mixture containing corn mash as the organic material for fermentation.

Three hundred grams of mash per cup were prepared. Ground wet corn (104.7 g) was added to a 500 mL round bottom mash cup, followed by 195.3 g of tap water. The mash pH was checked and adjusted between 5.8 and 6.0. The flask was heated to 80° C. in the mash bath and maintained at 80° C. for 90 min. A 57 μL aliquot of Alpha amylase (Termamyl Classic, Novozymes®, Denmark), was added to each cup. The mash was continuously mixed at 200 rpm for the time. The saccharification was conducted once the mash had cooled down to 32ºC. Glucoamylase (San Super 360L, Novozymes®, Denmark) was added (65 μL) and the mash maintained for 60 min at 200 rpm. The content of the mash cup was transferred to a 500 mL air locked Erlenmeyer flask. The pH was checked and adjusted to 5 with sulphuric acid.

The genetically modified yeast was grown from a cryogenic tube kept at −80° C. on a YPD agar plate and incubated for up to 48 hours at 32° C. From the YPD plate one colony was inoculated into a 5 mL culture tube with YPD broth and kanamycin and incubated overnight for approximately 16 hours in order achieve an optimal cells concentration of 10⁷ cells/mL. 260 μL from the culture tube was aliquoted into each fermentation flask. Urea was prepared by weighing 5 g in a volumetric flask and water added to make up 50 mL and an aliquot of 1.90 mL was pipetted in each Erlenmeyer flask.

A control of Ethanol Red (ER) (no compounds dosed) and genetically modified yeast were grown in medium without hop acids, samples of genetically modified yeast was also treated with IsoStab® (BetaTec Hop Products, UK) at 150 ppm product.

The samples were incubated at 32ºC and 200 rpm and maintained for 66 hours. A sample from each flask was pulled at the end of the fermentation and analysed for sugars, alcohols and glycerol levels by HPLC.

HPLC is the current standard analytical procedure for monitoring ethanol production and uses an ion exclusion column, such as the Phenomenex® Rezex ROA, 300×7.8 mm. This technology utilizes several different separation modes (gel filtration, ion-exchange, and reversed phase) to separate all the fermentation components (sugars, organic acids, and alcohols) in one chromatographic separation. Crude samples of the bioethanol fermentation time points are diluted 1:2 using water and filtered through a syringe attached with a cotton ball and passed through a Phenex 0.45 μm syringe filter.

The ethanol HPLC testing standard was obtained from Bion Analytical, USA. Filtered aliquots of 8 μL were injected on a HPLC operating at a flow rate of 0.6 mL/min. The HPLC column was heated to 65° C. SecurityGuard™ cartridges are regularly changed every 100 runs. 0.005 N sulphuric acid in water is used in the autosampler needle wash to avoid bacterial contamination. All analyses were run on Agilent® 1260 II system, equipped with a 1260 MCT Column oven pump G7111B, 1260 Vialsampler G7129A, built in Degasser and a 1260 RI detector G7162A. Data was collected using Agilent OpenLab Software version 2.3. A single dimension of Rezex ROA Organix Acid H+(8%) column (Phenomenex Inc.) was used: 300×7.8 mm. Aqueous mobile phase was 0.005 N sulfuric acid in water.

After 66 hours of fermentation, the % ethanol by volume in ER control fermentations (n=2, lacking hop acids) was 14.60; the % ethanol by volume in Variant G fermentations without hop acid (n=4) was 14.39; and the % ethanol by volume in Variant G with 150 ppm IsoStab® (n=4) was 14.85. This demonstrates that although Variant G produced less ethanol than the native strain in the absence of hop acids, addition of hops improved ethanol production by 3.15% (from −1.44% to 1.71%).

### Example 3: Molasses Fermentation

Genetically modified yeast were used in a fermentation mixture containing molasses as the organic material for fermentation.

The required volume of cane molasses must for each experiment was prepared by diluting syrup molasses (˜70° brix) to 20° Brix with tap water, this is equivalent to 1 part syrup molasses and 3 parts of tap water.

The pH was checked (initial pH was around 5.2) and adjusted to pH 5 by the addition of sulphuric acid. The molasses must was transferred into 500 mL air locked Erlenmeyer flasks to give a final volume of 300 mL.

The genetically modified yeast was grown from the master culture stored at −80° C., by streaking a colony onto a YPD agar plate which contained kanamycin and incubating it for up to 48 hours at 32° C. From the YPD plate, a colony was inoculated into 5 mL culture tube with YPD broth and kanamycin and incubated overnight for approximately 16 hours in order achieve an optimal cells concentration of 10⁷ cells/mL. 260 μL was aliquoted into each fermentation flask from the culture tube.

The urea was prepared by weighing 5 g in a 50 ml volumetric flask and made up to volume with water and aliquoted 1.90 mL urea in each Erlenmeyer flask.

A control of PE-2 (no compounds dosed) and genetically modified yeast were grown in medium without hop acids, samples of genetically modified yeast was also treated with LactoStab® (BetaTec Hop Products, UK) at 13.5 ppm product.

The samples were incubated at 32° C. and 200 rpm for 64 hours. A sample from each flask was pulled at the end of the fermentation and analysed for sugars, alcohols and glycerol levels by HPLC as described in Example 2.

Molasses fermentation using standard small scale fermentation mimicking large scale plant conditions showed that Variant Q and Variant W were are significantly better in producing ethanol as compared to the PE-2 strain, both with or without hop acids in the culture medium.

In Variant Q tests, after 66 hours of fermentation, the % ethanol by volume in PE-2 control fermentations (n=2, lacking hop acids) was 8.4; the % ethanol by volume in Variant Q fermentations without hop acid (n=4) was 8.51; and the % ethanol by volume in Variant Q with 13.5 ppm LactoStab® (n=4) was 8.52. This demonstrates that Variant Q had an average increase of 1.31% as compared to PE-2 in the absence of hop acids and an increase of 1.43% when fermented in the presence of hop acids.

In Variant W tests, after 66 hours of fermentation, the % ethanol by volume in PE-2 control fermentations (n=2, lacking hop acids) was 8.75; the % ethanol by volume in Variant W fermentations without hop acid (n=4) was 8.96; and the % ethanol by volume in Variant W with 13.5 ppm LactoStab® (n=4) was 9.00. This demonstrates that Variant W had an average increase of 2.40% as compared to PE-2 in the absence of hop acids and an increase of 2.86% when fermented in the presence of hop acids.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A genetically modified yeast strain comprising a S. cerevisiae comprising a modification of at least one gene encoding mitochondrial release factor 1 (MRF1), YOR292C, or YGR190C, wherein the modification disrupts expression of a protein from the gene.

2. The genetically modified yeast strain of claim 1, wherein the modification is of a nucleic acid sequence of the gene.

3. The genetically modified yeast strain of claim 2, wherein the modification comprises an insertion into a coding portion of the gene.

4. The genetically modified yeast strain of claim 2, wherein the modification comprises deletion of all or a portion of the gene.

5. The genetically modified yeast strain of claim 1, wherein the modification is not of a nucleic acid sequence of the gene.

6. The genetically modified yeast strain of claim 1, wherein the S. cerevisiae comprises a modification of at least two genes encoding MRF1, YOR292C, or YGR190C.

7. The genetically modified yeast strain of claim 1, wherein a gene encoding MRF1, absent modification, has a nucleic acid sequence at least 90% identical to that of SEQ ID NO: 1, or encodes a protein having an amino acid sequence at least 90% identical to that of SEQ ID NO: 2.

8. The genetically modified yeast strain of claim 1, wherein the gene encoding YOR292C, absent modification, has a nucleic acid sequence at least 90% identical to that of SEQ ID NO: 3, or encodes a protein having an amino acid sequence at least 90% identical to that of SEQ ID NO: 4.

9. The genetically modified yeast strain of claim 1, wherein the gene encoding YGR190C, absent modification, has a nucleic acid sequence at least 90% identical to that of SEQ ID NO: 5, or encodes a protein having an amino acid sequence at least 90% identical to that of SEQ ID NO: 6.

10. The genetically modified yeast strain of claim 1, comprising an ER yeast strain comprising a modification in the gene encoding YGR190C.

11. The genetically modified yeast strain of claim 1, comprising a PE-2 yeast strain comprising a modification in the gene encoding YOR292C.

12. The genetically modified yeast strain of claim 1, comprising a PE-2 yeast strain comprising a modification in the gene encoding MRF1.

13. A fermentation mixture comprising:
a genetically modified yeast strain of claim 1;
an organic material for fermentation; and
a hop acid.

14. The fermentation mixture of claim 13, wherein the hop acid comprises an alpha acid or an iso-alpha acid.

15. The fermentation mixture of claim 13, where in the hop acid comprises a beta acid.

16. The fermentation mixture of claim 14, wherein the hop acid comprises a tetra-hydro iso-alpha acid (THIAA), a hexa-hydro iso-alpha acid (HHIAA), or a combination thereof.

17. A fermentation mixture comprising:
a genetically modified yeast strain of claim 1; and
an organic material for fermentation.

18. A fermentation method comprising fermenting a fermentation mixture of claim 1, for a time sufficient to produce between 0.5% and 4% more ethanol than fermenting an otherwise identical fermentation mixture lacking the hop acid.

19. A fermentation method comprising fermenting a fermentation mixture of claim 1, for a time sufficient to produce between more ethanol than fermenting an otherwise identical fermentation mixture using an unmodified native yeast strain that was modified to produce the genetically modified yeast strain.

20. A fermentation method comprising fermenting a fermentation mixture of claim 19, for a time sufficient to produce more ethanol than would be obtained by fermenting an otherwise identical fermentation mixture using an unmodified native yeast strain that was modified to produce the genetically modified yeast strain.
